(19) United States
(12) Patent Application Publication
(10) Pub. No.: US 2024/0087705 A1
(43) Pub. Date: Mar. 14, 2024

(54) PREDICTIVE DOSAGE SYSTEM AND METHOD FOR BEHAVIORAL HEALTH TREATMENTS

(71) Applicant: Rethink Autism, Inc., New York, NY (US)

(72) Inventors: Daniel A. Etra, New York, NY (US); Eran Rosenthal, New York, NY (US); Jamie C. Pagliaro, New York, NY (US); David J. Cox, New York, NY (US)

(21) Appl. No.: 18/371,654

(22) Filed: Sep. 22, 2023

Related U.S. Application Data
(63) Continuation-in-part of application No. 17/174,962, filed on Feb. 12, 2021, now abandoned.
(60) Provisional application No. 62/976,675, filed on Feb. 14, 2020.

(51) Int. Cl. G16H 20/10; G16H 50/70

(52) U.S. Cl. CPC G16H 20/10; G16H 50/70

(57) ABSTRACT

A computer-implemented automated monitoring and behavioral health treatment recommendation method comprising: receiving a set of data from a patient sensor and a set of data from a caregiver for a patient; computing, via a processor, an behavioral health dosage for the patient; correlating, via a processor, the behavioral health dosage to a corpus of behavioral health dosage treatment data; and calibrating the behavioral health dosage, via processor; in light of a cohort of similar patient behavioral health dosages and; measuring a progress metric towards a set treatment goal of the patient, via the processor.

### CROSS REFERENCE TO RELATED APPLICATIONS

This application is a continuation in part application of U.S. application Ser. No. 17/174,962, filed Feb. 12, 2021 and claims the benefit of U.S. Provisional Application No. 62/976,675, filed Feb. 14, 2020, and the entire contents of both filings which are incorporated by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and, in the drawings, that form a part of this document: Copyright 2020-2023, Rethink Autism Inc., All Rights Reserved

### TECHNICAL FIELD

This document pertains generally, but not by way of limitation, to optimizing assessment and treatment of individuals requiring counseling and specific treatment regimens when suffering from autism or other behavioral and mental health conditions

### BACKGROUND

### Autism Spectrum Disorders

Autism is a complex neurological and developmental disorder with behavior phenotypes including impairments in social skills, communication difficulties, and repetitive, rigid, restricted behaviors. Across the nation, autism occurs in an estimated one in 36 children; boys are nearly four times more likely to be diagnosed with autism than girls, and autism can affect any ethnicity. Autism is identified as a deficit in communication that can range from a delay to total lack of verbal communication, restricted interests, and inconsistencies in responding. (Other impairments include atypical development in responding, vocalization, play, initiation, and response to name.)

Autism prognosis is dependent on the early identification of the disorder, thus supporting the continued need to begin evidence-based treatment as early as possible. Early intervention is a critical time in development in which the modifiability of developmental trajectory is high, thus allowing learners the opportunity to move toward a normative developmental trajectory. As the gap in development increases between a child's age and a child's development, remediation of concerns become more challenging.

### Aba Definitions and Treatment Models

ABA is an acronym for Applied Behavior Analysis, and it is often described as the “gold standard” for autism treatment. Applied Behavior Analysis (ABA) is a system of autism treatment based on behaviorist theories which, simply put, state that desired behaviors can be taught through a system of carefully delivered rewards, reinforcements, and consequences. ABA can be thought of as applying behavioral principles to behavioral goals and carefully measuring the results.

While the idea of using rewards and consequences to teach behavior is probably as old as human civilization, the idea of carefully applying rewards and consequences to achieve specific, measurable goals is relatively new.

### How Aba Works

Discrete trials ABA is still in use in some settings, and for some children. Other forms of ABA, however, are becoming increasingly popular such as natural environment training and incidental teaching. In addition, rather than providing 1:1 therapy in a classroom or office, many therapists are now administering ABA in natural settings such as playgrounds, cafeterias, and community locations. This approach makes it easier for children to immediately use the skills they learn in a real-world situation.

There are two commonly accepted models of ABA treatment; Comprehensive and Focused. Both models have variations in program dimensions such as peer involvement, parent involvement or delivery approach (i.e. structured vs naturalistic.) Comprehensive ABA treatment is generally delivered in large dosages ranging from 25-40 hours per week and addresses a large number of developmental domains including communication, social, behavioral and academic. The length of treatment varies, but at minimum treatment should occur for two years.

Comprehensive ABA treatment targets skill acquisition across multiple developmental domains by increasing communication, social, emotional, adaptive and cognitive functioning skills as well as reducing maladaptive behaviors that impact learning, client health and safety, family and community safety and functional independence. The cost of this program can exceed $50,000 per year, but it has also been suggested that future healthcare cost can be greater over longer periods of time if ABA treatment is not provided.

A second treatment model is Focused ABA Treatment. Different from Comprehensive ABA, Focused ABA treatment addresses a limited number of behavioral targets. It is optimal for individuals who need only to acquire a limited number of pivotal skills or for those who have an acute problem behavior that affects the health and safety of themselves or others. Dosage of treatment will vary, but on average occurs at 10-25 hours per week.

A problem identified in this treatment regime is that no set of assessments and treatment schedules have been normalized between the payer (insurance) community and the provider (treatment) community. There are also large discrepancies between the services provided between various providers in the community as well as the levels of services authorized by the payer communities; this can be quantified as a larger problem.

### BRIEF SUMMARY

The inventors of this solution have further recognized the need to incorporate active diagnosis and treatment into the homes of the patients. The solution includes a Virtual Medical Home (VMH), a solution variant that melds various facets of advanced health technologies into an integrated ecosystem for comprehensive personal behavioral and mental healthcare. Central to this system are its mental health sensors and other behavioral inputs, telehealth capabilities, and a sophisticated real-time diagnostic toolkit with a keen emphasis on mental health evaluations.

The mental health sensors range from various diagnostic inquiry tools for the patient or their care giver to a suite of biomedical wearable devices calibrated for continuous monitoring of parameters related to indicating a patient's mental state. By tracking critical physiological parameters such as, eye tracking, heart rate, blood pressure, oxygen saturation, body temperature and other parameters, these devices integrate via wireless data transmission. An embedded algorithm within the system monitors this influx of data, set to dispatch alerts upon detecting deviations from preset norms. These crucial alerts are channeled to the caregiver's interactive interface, the patient's attention (if desired) and the healthcare provider's dashboard, allowing for prompt intervention if necessary. Additionally a set of nudges or alerts can be provided to alert a caregiver to issue corrective actions if warranted for intervention.

The VMH telehealth module is designed with an intuitive graphical user interface (GUI) that functions as a bridge between patients and healthcare providers. Beyond mere video consultations, this interface is deeply integrated with a robust backend database system. This repository not only holds critical data such as a patient's medical history and current medications but also dynamically updates with insights from the real-time diagnostic tools, ensuring that practitioners always have a 360-degree view of the patient's health landscape.

The VMH comprises an advanced real-time diagnostic platform for mental and behavioral health. This platform is constructed upon three pillars:
Behavioral Tracking/Management Modules. Using machine learning the module data accumulated from both user inputs and physiological indicators is compared against anticipated values, offering insights into a patient's emotional state of well-being. Fluctuations, patterns, and trends are identified, mapped, and analyzed to gauge mood stability and potential triggers.
Interactive Cognitive Behavioral Therapy Modules: This software suite presents users with a series of structured exercises and interventions grounded in established psychotherapeutic principles. Designed to be interactive, these modules adapt in real-time, tailoring their guidance according to user responses and feedback.
AI-driven Analysis Framework: This framework dives deep into the vast array of data the system accrues. From sleep patterns to speech nuances, the AI system is trained to pick up subtle indicators that supplement practitioner or human observation. Potential areas of concern are flagged, and, if necessary, the system can recommend professional intervention or tailored therapeutic exercises. Using the VMH, the inventors of this solution have recognized the advantages of providing a standardized set of assessments for both Focused ABA and Comprehensive ABA filings via the VMH. The inventors have also recognized the advantages of providing a solution that allows a normalization of treatment both for providers and payers to allow for easy approval, easy delivery, and easy evaluation of the quality of commonly prescribed services. The inventors have also recognized the advantages of providing the assessments and the approval of treatments requested via a mobile device to optimize the ready gathering, dissemination and decision making by providers and the approval of prescribed treatment. In addition, the inventors have also recognized that using a corpus of recommendations and treatment data, and training the solution, using a computer system, to optimize its recommendations and receiving the feedback allows the solution to make better and better recommendations.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an Assessment Flow 100 in accordance with one example of the solution.

FIG. 2 illustrates a Networked Solution 200 in accordance with one example of the solution.

FIG. 3 illustrates a Solution processing environment 300 in accordance with one example of the solution.

FIG. 4 illustrates a Solution block diagram 400 in accordance with one example of the solution.

FIG. 5 illustrates a Solution machine 500 in accordance with one example of the solution.

FIG. 6 illustrates a Solution machine-learning program 600 in accordance with one example of the solution.

FIG. 7 illustrates an Introduction Screen User Interface 700 in accordance with one example of the solution.

FIG. 8 illustrates an Open Assessment User Interface 800 in accordance with one example of the solution

FIG. 9 illustrates an Assessment Choice User Interface 900 in accordance with one example of the solution.

FIG. 10 illustrates a Treatment Model Input User Interface 1000 in accordance with one example of the solution.

FIG. 11 illustrates a Planning Data User Interface 1100 in accordance with one example of the solution.

FIG. 12 illustrates a Current Status User Interface 1200 in accordance with one example of the solution.

FIG. 13 illustrates an Environmental Considerations User Interface 1300 in accordance with one example of the solution.

FIG. 14 illustrates a Clinical Data User Interface 1400 in accordance with one example of the solution.

FIG. 15 illustrates a Result and Recommendation User Interface 1500 in accordance with one example of the solution.

FIG. 16 illustrates a Results Summary User Interface 1600 in accordance with one example of the solution.

FIG. 17 illustrates a Comprehensive Report User Interface 1700 in accordance with one example of the solution.

FIG. 18 illustrates a charting interface example 1800 in accordance with one example of the solution

FIG. 19 illustrates an aspect of the subject matter in accordance with one embodiment

FIG. 20 illustrates an aspect of the subject matter in accordance with one embodiment.

FIG. 21 illustrates an aspect of the subject matter in accordance with one embodiment.

FIG. 22 illustrates an aspect of the subject matter in accordance with one embodiment.

### DETAILED DESCRIPTION

Embodiments of this solution may be implemented in one or a combination of hardware, firmware and software. Embodiments may also be implemented as instructions stored on a computer-readable storage device, which may be read and executed by at least one processor to perform the operations described herein. A computer-readable storage device may include any non-storing information in a form readable by a machine (e.g., a computer). For example, a computer-readable storage device may include read-only memory (ROM), random-access memory (RAM), magnetic disk storage media, optical storage media, flash-memory devices, cloud servers or other storage devices and media. Some embodiments may include one or more processors and may be configured with instructions stored on a computer-readable storage device. The following description and the referenced drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The present solution automates and improves the efficacy, the predictability of treatment and efficiency of insurance claims around autism and other behavioral and mental health conditions. The present solution integrates adaptive treatment strategies with real-time monitoring to optimize individual-level patient outcomes and responses. At its foundation, the solution uses a learning system that addresses the inherent variability and non-static nature of various mental and behavioral health conditions.

The architecture of the solution is fundamentally based on continuous data acquisition coupled with decision-making algorithms. Through the use of wearable sensors, mobile device telemetry, and digital self-report mechanisms, the solution systems gather extensive data on physiological, behavioral, and self-reported psychological parameters. These parameters might include but are not limited to, metrics like heart rate variability, skin conductance, device interaction patterns, physical movement, and direct user input about their current emotional or cognitive state. Eye tracking is also a metric that is also very useful in treating patients. Eye tracking can offer additional insights into the behavioral patterns and cognitive processes of individuals. Here's how eye tracking can be relevant: Social cues: Eye tracking can provide data on where an individual is focusing when they are shown pictures or videos featuring social scenarios. Assessing attentive patterns: eye tracking can provide feedback on what captures the individual's attention, how long they can maintain focus, and what might be causing distractions. And evaluation of current treatment outcomes: Eye tracking can serve as an objective measurement tool to evaluate the effectiveness of ABA interventions over time. By comparing eye tracking data before and after an intervention, therapists can assess whether behavioral changes have occurred and if the individual has improved in targeted areas such as attention or social interaction

Within the solution's framework, the real-time data is subjected to systematic computational analysis. Using complex algorithms that are grounded in statistical and machine learning methods, the system processes the incoming data streams to identify markers that might signal a change or potential degradation in an individual's mental state or behavioral state. These algorithms are not just responsive but predictive, aiming to identify and act upon potential mental or behavioral health shifts before they become pronounced.

The response mechanism within the solution is adaptive. It employs a pre-determined set of intervention strategies, which are deployed based on the specific trigger or combination of triggers detected. The uniqueness of the solution lies in its capacity to choose an intervention tailored to the immediate needs of the individual and the context. For instance, if the system identifies heightened stress levels during a user's typical bedtime, it might recommend to a caregiver that the patient work through a guided relaxation exercise . . . .

Moreover, the interventions can vary in intensity and mode of delivery. On detecting less severe changes, the solution might offer cognitive behavioral exercises, diversionary exercises or mindfulness practices for the caregiver to execute with the patient. In cases where the detected changes are of higher concern or persist despite initial interventions, the system might escalate its response, such as alerting a designated mental health professional, initiating a telehealth session or suggesting to a caregiver that the individual should seek immediate professional support.

Another significant technical feature of the solution system is the continuous feedback loop. Post-intervention data continues to flow into the system, allowing it to gauge the effectiveness of the chosen strategy. Over time, and with accumulating data, the solution refines its intervention selection process. Through this iterative mechanism, the system continually optimizes its decision-making algorithms to improve the specificity and efficacy of interventions for the user.

Data security and privacy are of paramount importance in the solution systems. Given the sensitivity of mental and behavioral health data, robust encryption techniques and secure data transmission protocols are integrated into the system architecture. Additionally, there's a balance to be struck between data storage and real-time processing. While some data may be stored for retrospective analysis, much of the solution processing can occur on-device (e.g. edge computing), ensuring that personal data doesn't always need to be transmitted to centralized servers in the cloud.

Furthermore, for the solution system to be truly effective, it needs to be integrated within a broader healthcare framework. This means creating interfaces with existing electronic health record systems, ensuring that healthcare professionals can input or access relevant information such as measured conditions and triggering thresholds of the system solution when needed. Also, the transparency and interpretability of the decision-making algorithms is crucial. While black-box algorithms might offer predictions, educating practitioner and other healthcare professionals as to the data triggers, allows the professionals serving the patient a quick pathway to understand the basis for any automated recommendation or alert.

### Autism Spectrum Disorders

Autism is a complex neurological and developmental disorder with behavior phenotypes including impairments in social skills, communication difficulties, and repetitive, rigid, restricted behaviors. Across the nation, autism occurs in an estimated one in 36 children; boys are nearly four times more likely to be diagnosed with autism than girls, and autism can affect any ethnicity. Autism is identified as a deficit in communication that can range from a delay to total lack of verbal communication, restricted interests, and inconsistencies in responding. Other impairments include atypical development in responding, vocalization, play, initiation, and response to name.

Autism prognosis is dependent on the early identification of the disorder, thus supporting the continued need to begin evidence-based treatment as early as possible. Early intervention is a critical time in development in which the modifiability of developmental trajectory is high, thus allowing learners the opportunity to move towards a normative developmental trajectory. As the gap in development increases between a child's age and a child's development, remediation of concerns become more challenging. By identifying developmental delays early in the child's life, one may decrease future risk of other medical and behavioral conditions, and therefore this surveillance should be routine and include specific inquiries related to the core symptoms of autism.

Autism is a chronic disorder in which on-going medical and social management of symptoms may be lifelong and is a necessary aspect of care. Children diagnosed with autism have shown improved health and developmental outcomes through the use of early, intensive and direct evidence-based treatment.

### Comorbidity

Because of the complexity of autism, comorbid conditions are common; however, these comorbidities may be difficult to detect given the social and communication impairments specific to autism. Autism is often accompanied with a range of coexisting behavioral difficulties including aggression, self-injury, hyperactivity, and obsessive-compulsive disorders. Individuals with autism may have frequent co-occurring medical conditions that increase mortality rates amongst this population. For example, seizures are prevalent in nearly 20-25% of the autism population. Other conditions include anxiety, disrupted sleep, immune dysregulation, allergies, gastrointestinal problems, metabolic abnormalities, and dysautonomia. Due to the extreme heterogeneity present in autism spectrum disorders, treatment of comorbid conditions varies, and may be complex and continuous, requiring ongoing collaboration and care with a primary care physician.

With the prevalence occurring in 50-80% of patients with autism, disruptive sleep remains one of the most significant and problematic health concerns amongst parents and professionals treating autism. Additionally, sleep disturbances in children are a key symptom that assists in the identification of autism in young children under the age of two. Sleep difficulty is not specifically restricted to just early childhood but may also be a lifelong concern for families and as such may result in increased parental stress. In young children with autism, families report sleep behavior concerns specifically related to sleep anxiety, bedtime resistance, and night awakenings.

The present solution automates and improves the efficacy of the ABA treatments prescribed, improves the predictability of treatment by providing statistical output and guidelines based on various data and studies and improves efficiency and predictability of authorization requests and payments of insurance claims.

### General Description of the Aba Treatment Regime

ABA is considered an evidence-based best practice treatment by the US Surgeon General and by the American Psychological Association. “Evidence based” means that ABA has passed scientific tests of its usefulness, quality, and effectiveness. ABA therapy includes many different techniques. All of these techniques focus on antecedents (what happens before a behavior occurs) and on consequences (what happens after the behavior). More than 20 studies have established that intensive and long-term therapy using ABA principles improves outcomes for many but not all children with autism. “Intensive” and “long term” refer to programs that provide 25 to 40 hours a week of therapy for 1 to 3 years. These studies show gains in intellectual functioning, language development, daily living skills and social functioning. Studies with adults using ABA principles, though fewer in number, show similar benefits.

There are two commonly accepted models of ABA treatment; Comprehensive and Focused. Both models have variations in program dimensions such as peer involvement, parent involvement or delivery approach (i.e. structured vs naturalistic) Comprehensive ABA treatment is generally delivered in large dosages ranging from 25-40 hours per week and addresses a large number of developmental domains including communication, social, behavioral and academic. The length of treatment varies, but at minimum treatment should occur for two years. Comprehensive ABA treatment targets skill acquisition across multiple developmental domains by increasing communication, social, emotional, adaptive and cognitive functioning skills as well as reducing maladaptive behaviors that impact learning, client health and safety, family and community safety and functional independence. The cost of this program can exceed $50,000 per year, but it also has been suggested that future healthcare cost can be greater over longer periods of time if the treatment is not provided.

A second treatment model is Focused ABA Treatment. Different from Comprehensive ABA, Focused ABA treatment addresses a limited number of behavioral targets. It is optimal for individuals who need only to acquire a limited number of pivotal skills or for those who have an acute problem behavior that affects the health and safety of themselves or others. Dosage of treatment will vary, but on average occurs at 10-25 hours per week.

### Medical Necessity Assessment Criteria and its Variances

The various insurers and state agencies publish varied requirements to determine medical necessity. For example, here below is a set of AETNA criteria that would be used to validate ABA therapy claims and be used by the solution to validate medical necessity.

There must be a diagnosis of a condition on the Autism Spectrum (ICD-9: 299 through 299.9; ICD-10: F84 through F84.9)

There are identifiable target behaviors having an impact on development, communication, interaction with typically developing peers or others in the child's environment, or adjustment to the settings in which the child functions, such that the child cannot adequately participate in developmentally appropriate essential community activities such as school. The ABA is not custodial in nature (which AETNA defines as care provided when the member “has reached the maximum level of physical or mental function and such person is not likely to make further significant improvement” or “any type of care where the primary purpose of the type of care provided is to attend to the member's daily living activities which do not entail or require the continuing attention of trained medical or paramedical personnel.”) Plan documents may have variations on this definition and need to be reviewed.

Parent(s) (or guardians) must be involved in training in behavioral techniques so that they can provide additional hours of intervention.

There is a time limited, individualized treatment plan developed that is child-centered, strengths-specific, family-focused, community-based, multi-system, culturally-competent, and least intrusive; where specific target behaviors are clearly defined; frequency, rate, symptom intensity or duration, or other objective measures of baseline levels are recorded, and quantifiable criteria for progress are established; describing behavioral intervention techniques appropriate to the target behavior, reinforces selected, and strategies for generalization of learned skills are specified; and there is documentation of planning for transition through the continuum of interventions, services, and settings, as well as discharge criteria.

There is involvement of community resources to include at a minimum, the school district if the child is 3 or older, or early intervention if not.

Services must be provided directly or billed by individuals licensed by the state or certified board unless state mandates, plan documents or contracts require otherwise. If state mandates, plan documents or contracts allow authorization for services that are not directly provided by individuals licensed by the state or certified by the Behavior Analyst Certification Board, there must be supervision of the unlicensed or non-certified providers, unless state mandates, plan documents or contracts require otherwise. Supervision is to be documented and is defined as at least one hour of face-to-face supervision of the unlicensed or non-certified provider by a certified behavior analyst or licensed psychologist for each ten hours of behavioral therapy by the supervised provider, and at least one hour a month face-to-face, on-site with the patient.

As may be appreciated, criteria vary between insurance carriers and the various states, and thus the solution must ingest the criteria of many different providers and states to reliably help a clinician (provider) prescribe ABA therapy in the way best suited to the patient and to the payer (insurer.)

Behavioral tracking/management modules are a type of technology that uses machine learning to track and analyze human behavior. These modules can be used to monitor a variety of behaviors, including:
Emotional states: These modules can track changes in mood, stress levels, and anxiety.
Physical activity: These modules can track steps taken, calories burned, and heart rate.
Sleep patterns: These modules can track sleep duration, quality, and disturbances.
Dietary habits: These modules can track food intake, calories consumed, and nutrients.
Social interactions: These modules can track the frequency and quality of social interactions.

The data collected by behavioral tracking/management modules can be used to identify patterns and trends in behavior. This information can be used to improve mental and physical health by:
Identifying early warning signs of mental health problems: These modules can help to identify changes in mood, stress levels, and sleep patterns that may be early warning signs of mental health problems such as depression, anxiety, and bipolar disorder.
Monitoring the effectiveness of treatment: These modules can be used to monitor the effectiveness of treatment for mental and behavioral health problems. For example, a module that tracks sleep patterns can be used to see if a patient's sleep is improving after starting medication or therapy.
Providing personalized feedback and interventions: These modules can be used to provide personalized feedback and interventions to help people improve their mental and physical health. For example, a module that tracks physical activity can provide feedback on how to increase physical activity levels.

The method of behavioral tracking/management modules work:
Data collection: The first step is to collect data on the behavior that is being tracked. This data can be collected in a variety of ways, such as through care giver observations self-report surveys, wearable devices, and environmental sensors.
Data processing: Once the data is collected, it needs to be processed in order to make it suitable for analytics and statistical modeling. Data processing often involves cleaning, transformations, handling missing values, and so forth
Data analysis: The processed data can then be analyzed to identify patterns and trends. This can be done using machine learning algorithms. This information can be used to improve mental and physical health.
Feedback and interventions: The results of the data analysis can be used to provide personalized feedback and interventions to help people improve their mental and physical health.

AI-driven Analysis Framework within the Virtual Medical Home System (VMH)

The VMH's AI-driven framework is predicated on the principle of deep data analysis. As the framework ingests and processes a plethora of data, it showcases the capability to decode intricate patterns within these data sets. One of the most distinguishing features of this framework is its ability to scrutinize diverse types of data—from quantifiable metrics like sleep patterns to qualitative aspects like speech nuances. By doing so, the system aligns itself with a multi-modal approach to healthcare, wherein both objective and subjective indicators of a patient's wellbeing are considered holistically.

Sleep patterns, for instance, offer invaluable insights into an individual's overall health and particularly their mental well-being. A consistent deviation in sleep cycle, duration, or quality can be indicative of underlying stressors, anxiety, depression, or other health concerns. The AI, with its robust algorithmic foundation, can identify these deviations, compare them against benchmark data, and forecast potential health risks.

Moreover, the analysis of speech nuances opens a realm of opportunities for understanding psychological and neurological conditions. Variations in speech speed, tone, frequency, and vocabulary usage can be predictors of conditions like stress, depression, or even degenerative diseases like Parkinson's. The subtleties that might go unnoticed or be deemed insignificant in manual assessments become quantifiable metrics within this AI framework, ensuring that no symptom, however minor, is overlooked.

It's vital to note that the AI system isn't intended to replace human observation but to supplement it. Practitioners, with years of training and experience, possess the intuitive capability to understand and interpret symptoms. However, human observation can sometimes be limited by subjectivity or oversight. The AI-driven framework counteracts these limitations by offering objective, quantified, and consistent data-driven insights, ensuring a balanced blend of human intuition and machine precision using all available machine and human captured data.

Another pivotal aspect of the VMH's AI framework is its proactive approach to healthcare. Rather than waiting for symptoms to manifest overtly, the system is engineered to flag potential areas of concern at their nascent stages. This early detection is pivotal in conditions where timely intervention can halt progression or even reverse the condition. Moreover, the granularity of the system's analysis means that it can tailor its recommendations to the individual. Whether it's suggesting professional intervention for more pronounced concerns or recommending specific therapeutic exercises for minor deviations, the system ensures that the treatment is personalized.

The VMH's recognition of the advantages of a standardized set of assessments, especially for both Focused ABA (Applied Behavior Analysis) and Comprehensive ABA filings, further underscores its commitment to streamlined and effective care. ABA, a cornerstone in therapeutic interventions, especially for individuals with autism, requires meticulous tracking and assessment. By offering standardized assessments via the VMH, practitioners are furnished with a consistent, reliable, and efficient tool to monitor progress, adjust therapeutic strategies, and ensure optimal outcomes for the patients.

By recognizing the strengths of both machine-driven analytics and human intuition, the VMH positions itself at the forefront of revolutionizing mental health and behavioral interventions.

### Treatment/Assessment Flow

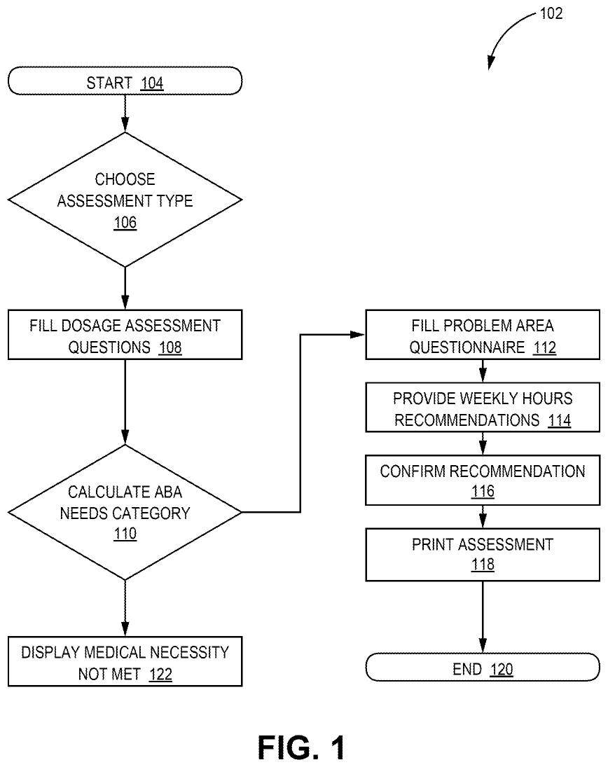

FIG. 1 shows The Assessment Flow 102 comprises a Start Step 104, a Choose Assessment 106, a Dosage Assessment Step 108, a Calculation of Needs 110, an Uncover Problem Area Step 112, a Recommendation Step 114, and a Confirmation step 116, an Assessment Output 118, an End 120, and a Medical Necessity Not Met Step 122. In this Assessment Flow 102, the user Choose Assessment 106 step must select a set of service options as the recommended dosage may differ based on whether the user is initially starting the treatment program or re-authorizing for new set of hours

Options comprise an Initial Service Authorization or a Service Re-authorization. The Dosage Assessment Step 108 uses a Questionnaire that has different sets of questions for each assessment type based on the impact on the needed dosage, and every question has a different weight. A weighted score for each question is based on the weight and the answer scoring. Once the Dosage Assessment Step 108 is complete, the process moves to an ABA Calculation of Needs 110 step where scoring of the question answers is performed resulting in three outcomes. A score in excess of 7 takes the solution to a subsequent comprehensive ABA step where the Uncover Problem Area Step 112 requires a subsequent questionnaires to fully define a set of comprehensive problems and treatments. A score between 4-7 results in a focused ABA where the Uncover Problem Area Step 112 requires a subsequent questionnaire to fully uncover a narrower set of focused problems and treatments for the subject of the Assessment Flow 102. A score less than 4 will result in a Medical Necessity Not Met Step 122 being completed. The Uncover Problem Area Step 112 comprises asking a user to answer questions related to the severity of each problem are, which along with the Dosage Category will determine the number of hours needed for each area. The Assessment Flow 102 also utilizes a machine learning step at the end as to whether a provider or payer agrees with the recommendation and the Assessment Output 118

Additional optional steps comprise a) Fine-tune hours recommendations for the different skills/behaviors categories b) receive recommendations and alerts when the solution algorithms identify an outlier, c) identify and isolate non-clinical factors influencing clinician recommendations, d) recommendations for improving insurance coverage, and e) monitor overall clinician agreement with system recommendations as an indicator of process performance and source of feedback for adjustments to process and questionnaire.

A non-limiting example of the types of data metrics is incorporated in the solution is listed by primary groups of cohorts and other segmentation fields. The solution also allows filtration of results via this set of data fields.

Patient Data Fields Collected and Coordinated.
Age Cohorts
Age at start of treatments
Current age
Utilization Cohorts
Utilized Hours/Authorized Hours
Utilized Hours/Recommended Hours
Severity Cohorts
Treatment Cohorts
Comprehensive vs. Focused ABA
MNA Recommended Hours
Other assessments
By Curriculum Area
By Gender
By Payer
By Individual Payer/Payer Group
Commercial vs. Public vs. Private Pay

Another view of the solution's data fields that are gathered on an exemplar patient. The patient is recorded by observation by the professional as well as by the parents of the patient. In the table below, a sample of the types of standard therapy measurements are listed

As may be readily intuited, the solution can be expanded or contracted based on the sophistication or cohort that the individual is assigned to. The tables below represent examples of other data items to be captured for the ABA ecosystem. This set of data items includes the infrastructure data to filter by patient, by clinician (provider), by state, by payer, by practice and by ABA benchmarks.

The data fields below capture various aspects of the ABA therapy, comprising metrics of acquisition or improvement of new skills by the patient are captured as well as capturing the behavioral reduction metrics of the individual and the trends related to improving various behaviors distinct from the skills metrics. Both sets of metrics are generally used by both providers and payers to create and analyze unique sets of targets, objectives and plans.

| | | | |
|---|---|---|---|
| By | By | By | Bench- |
| Child | Provider | Practice | marks |

RATE OF SKILLS ACQUISITION
1. Skills Targets Mastered/Time
a) Elapsed time since start of treatment
b) Hours of therapy (billable hours)
2. Skills Objectives Mastered/Time
a) Elapsed time since start of treatment
b) Hours of therapy (billable hours)
3. Skills Goals Mastered/Time
a) Elapsed time since start of treatment
b) Hours of therapy (billable hours)

Clinical fields are also captured as well as performance metrics of the patient to assist in justifying the appropriateness of therapy and to record the patient's engagement and successes using to measure improvement.

RATE OF BEHAVIOR REDUCTION
1. Behavior Objectives Mastered/Time
a) Elapsed time since start of treatment
b) Hours of therapy (billable hours)
2. Behavior Goals Mastered/Time
a) Elapsed time since start of treatment
b) Hours of therapy (billable hours)

Insurer fields are also captured to track utilization of the approved/used hours under MNA assessment.

UTILIZATION
1. Hours Used/Hours Authorized
2. Hours Used/Hours Recommended by MNA

Analytic and variance data fields are also provided as feedback to the insuring entity as well as benchmark and other outcomes for the purposes of tracking patient and therapy process and progress.

VARIANCE FROM MNA RECOMMENDATION
1. (Hours Requested − Hours Recommended)/Hours Recommended
2. (Absolute Value of (Hours Requested −
Hours Recommended))/Hours Recommended
Outputs
Calculated Statistics (and vs. Benchmarks)
Time Series and Trend lines (and vs. Benchmarks)
Scatter Plots

### Dosage Calculator Operating Environments

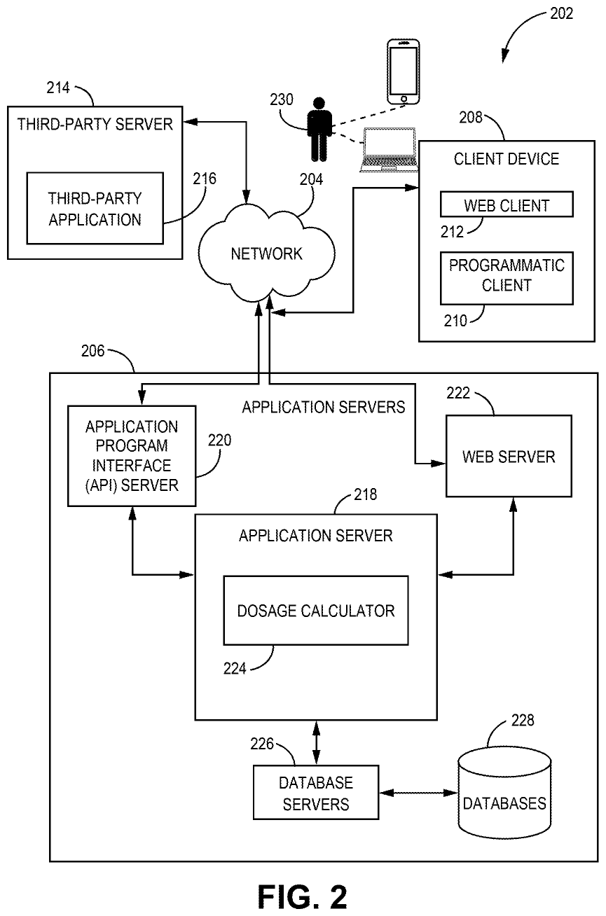

FIG. 2 is a diagrammatic representation of a networked computing environment 200 in which some examples of the present solution may be implemented or deployed.

One or more application servers 206 provide server-side functionality via a network 204 to a networked user device, in the form of a client device 208 that is accessed by a user 230. A web client 212 (e.g., a browser) and a programmatic client 210 (e.g., an “app”) are hosted and execute on the web client 1910.

An Application Program Interface (API) server 220 and a web server 222 provide respective programmatic and web interfaces to application servers 206. A specific application server 218 hosts a Dosage Calculator system 224, which includes components, modules and/or applications.

The web client 212 communicates with the Dosage Calculator system Dosage Calculator 224 via the web interface supported by the web server 220. Similarly, the programmatic client 210 communicates with the Dosage Calculator system Dosage Calculator 224 via the programmatic interface provided by the Application Program Interface (API) to the Application Program Interface (API) server 220. The third-party application 216 may, for example, be a Provider or Payer third party system accepting treatment regimens or an insurance claim relating to a patient.

The application server 218 is shown to be communicatively coupled to database servers 226 that facilitates access to an information storage repository or databases 228. In an example embodiment, the databases 228 includes storage devices that store information to be published and/or processed by the Dosage Calculator 224.

Additionally, a third-party application 216 executing on a third-party server 214, is shown as having programmatic access to the Environmental Considerations User Interface 1302 application servers 206 via the programmatic interface provided by the Application Program Interface (API) server 220. For example, the third-party application 216, using information retrieved from the application server 218, may support one or more features or functions on a website hosted by the third party.

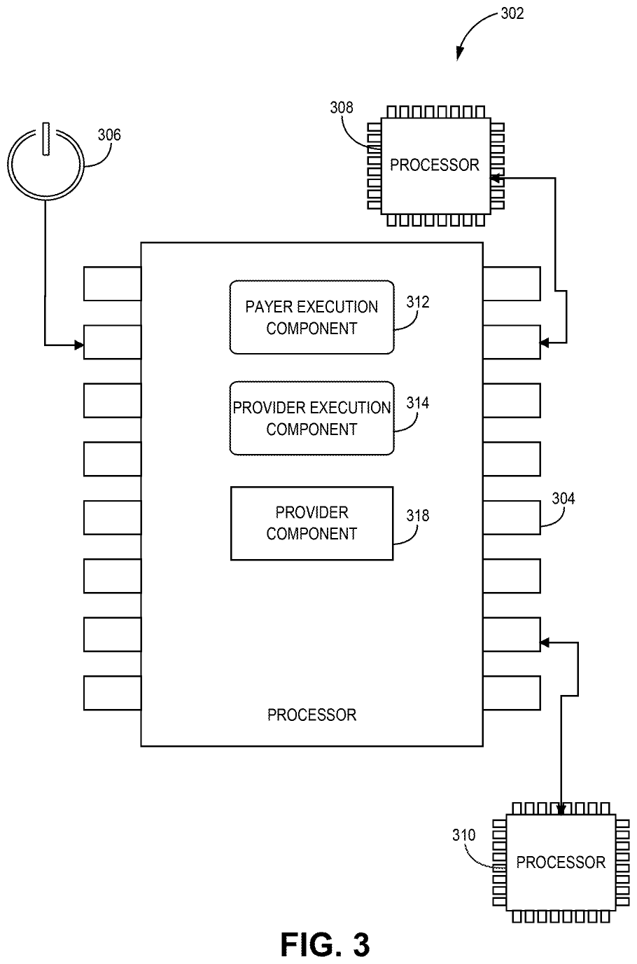

Turning now to FIG. 3, a diagrammatic representation of a multiprocessor processing environment 300 of the present solution is shown, which includes the Processor 308, the Processor 310, and a Processor 304 (e.g., a GPU, CPU or combination thereof).

The Processor 304 is shown to be coupled to a power source 306, and to include (either permanently configured or temporarily instantiated) modules, namely a Payer execution component 312, an Assessment Provider execution component 314 and a Provider Component 318. The Assessment execution component operationally generates Assessment Flow 102 and manages the user 230, the Payer Provider execution component 314 operationally generates Insurance claims and approvals, and the Provider execution component 314 operationally generates Clinical dosages and manages Provider Workflow. As illustrated, the Processor 304 is communicatively coupled to both the Processor 310 and Processor 308, and receives commands from the Processor 304, as well as commands from the Processor 308.

The following example is a non-limiting manner of enabling this solution. The solution also contemplates using other analogous hardware or software implementation than those explicitly mentioned.

### Technology and Infrastructure

This example solution is delivered over the Internet using virtual machines (VMs) for web and database servers, (e.g., AZURE web apps to host web applications, and AZURE blob storage for content storage. The solution deploys dynamically generated application features as well as derived or published content via various servers. The web applications are developed with any modern app development environment (e.g., Customer-facing solutions are developed in .NET Core, MVC Framework, C #, Angular, and SQL formats using Web 2.0 functionality standards.

### Data Centers

An example of the data center implementation uses 3^{rd} party data centers like those provided by GOOGLE, AMAZON, MICROSOFT vendors that comply with stringent data privacy requirement from the jurisdiction served by the platform.

### Servers and Content Hosting Technology

The solution hosts on INTEL or AMD processors using cloud-hosted server hardware on using various software formats for virtual machines. (e.g., Windows Server 2018.) Content, especially Video and other dynamic content is hosted by various media services (e.g. AZURE MEDIA SERVICE and BRIGHT COVE). All servers are set up in a high-availability fashion to ensure ultimate up-time, an active server takes in all the traffic and a warm backup server is on stand-by with continuous synchronization. This allows the solution to react to a server going down at any one point in time, the second server automatically kicks in without any manual intervention. Disaster recovery is also in place to ensure automatic failover in case of a data center outage.

### Solution Load Balancing for the Applications and Data

In this example of the solution, use of a 3^{rd} party (e.g., AZURE) hardware-based load balancing distributes end-user connections across a plurality of distributed servers. This enables greater resiliency, a balanced server load, with enhanced fault tolerance on the various applications deployed by this solution.

### Content Delivery Network

Content is held in a resilient blob storage that automatically replicates data to help guard against unexpected hardware failures. Content storage is triple-redundant with an option of geo-constrained redundant storage by jurisdictions that limit transfer of privacy related data. Content used in this context refers to content of all types and data of all types related to ABA treatment (e.g., educational literature, patient data and records, clinician data and records)

### Data Back-Up and Security

The solution uses multiple levels of backups to ensure data resiliencies. Full backups of data are created daily with transactional hourly backups. Copies of the backups are also physically or virtually sent to off-site facilities to ensure the highest level of protection and resilience for customer data.

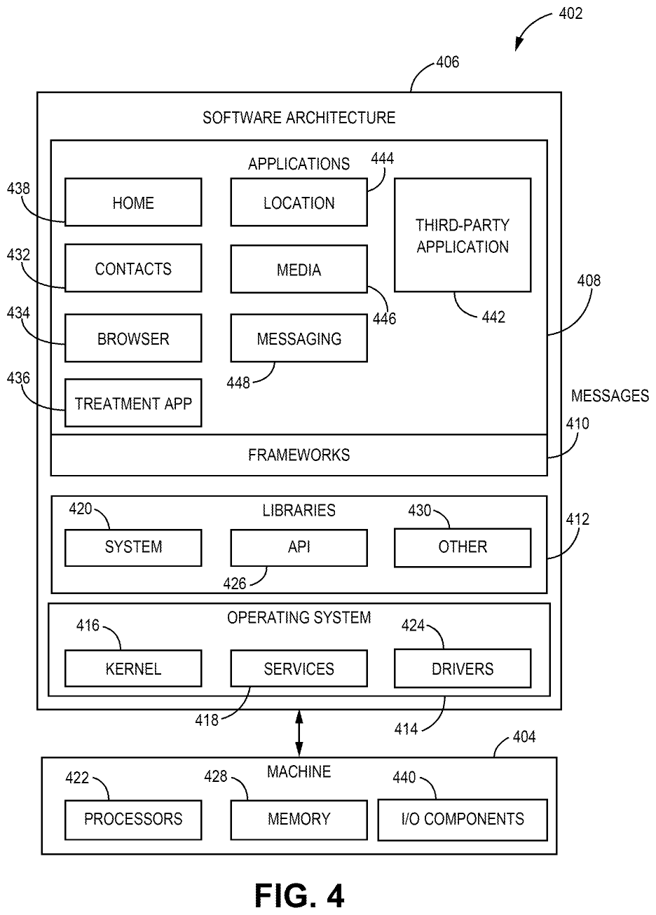

FIG. 4 is a solution block diagram 402 illustrating a software architecture 406 representative of the current solution, which can be installed on any one or more of the devices described herein. The software architecture 406 is supported by hardware such as a machine 404 that includes processors 422, memory 428, and I/O components 440. In this example, the software architecture 406 can be conceptualized as a stack of layers, where each layer provides a particular functionality. The software architecture 406 includes layers such as an operating system kernel 416, libraries 412, frameworks 410, and applications 408. Operationally, the applications 408 invoke API calls 452 through the software stack and receive messages 454 in response to the API calls 452.

The operating system 414 manages hardware resources and provides common services. The operating system 414 includes, for example, a kernel 416, services 418, and drivers 424. The operating system 414 acts as an abstraction layer between the hardware and the other software layers. For example, the kernel 416 provides memory management, Processor management (e.g., scheduling), component management, networking, and security settings, among other functionality. The services 418 can provide other common services for the other software layers. The drivers 424 are responsible for controlling or interfacing with the underlying hardware. For instance, the drivers 424 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, power management drivers, and so forth.

The libraries 412 provide a low-level common infrastructure used by the applications 408. The libraries 412 can include system libraries 420 (e.g., C standard library) that provide functions such as memory allocation functions, string manipulation functions, mathematic functions, and the like. In addition, the libraries 412 can include API libraries 426 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 412 can also include a wide variety of other libraries 430 to provide many other APIs to the applications 408.

The frameworks 410 provide a high-level common infrastructure that is used by the applications 408. For example, the frameworks 410 provide various graphical user interface (GUI) functions, high-level resource management, and high-level location services. The frameworks 410 can provide a broad spectrum of other APIs that can be used by the applications 408, some of which may be specific to a particular operating system or platform.

In an example of the solution, the applications 408 comprise a treatment app 436, a contacts application 432, a browser application 434 for recommended treatment or educational content, a location application 444 to capture where treatment is occurring, a media application 446, a messaging application 448, and a broad assortment of other applications such as a third-party application 442. The applications 408 are programs that execute functions defined in the programs. Various programming languages can be employed to create one or more of the applications 408, structured in a variety of manners, such as object-oriented programming languages (e.g., Objective-C, Java, or C++) or procedural programming languages (e.g., C or assembly language). In a specific example, the third-party application 442 (e.g., an application developed using the ANDROID™ or JQS™ software development kit (SDK) by an entity other than the vendor of the particular platform) may be mobile software running on a mobile operating system such as IOS™, ANDROID™, WINDOWS® Phone, or another mobile operating system. In this example, the third-party application 442 can invoke the API provided by the operating system 414 to facilitate functionality described herein.

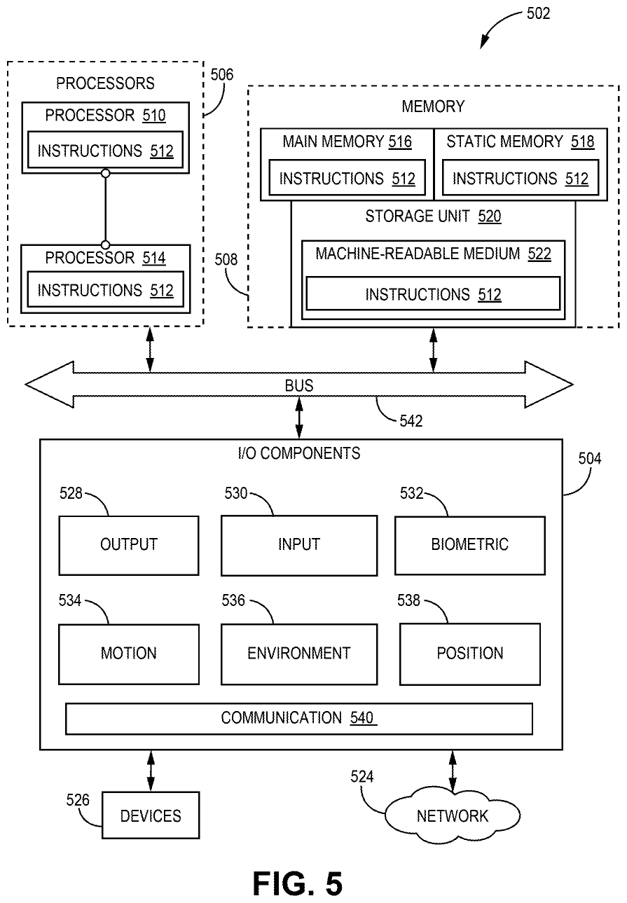

FIG. 5 is a diagrammatic representation of the solution machine 502 implementing the current solution within which instructions 512 (e.g., software, a program, an application, an applet, an app, or other executable code) for causing the machine 502 and its Processors 510, Processor 514 to perform any one or more of the methodologies discussed herein may be executed. For example, the instructions 512 may cause the machine 502 to execute any one or more of the methods described herein. The instructions 512 transform the general, non-programmed machine 502 into a particular machine 502 programmed to carry out the described and illustrated functions in the manner described. The machine 502 may operate as a standalone device or may be coupled (e.g., networked) to other machines. In a networked deployment, the machine 502 may operate in the capacity of a server machine or a client machine in a server-client network environment, or as a peer machine in a peer-to-peer (or distributed) network environment. The machine 502 may comprise, but not be limited to, a server computer, a client computer, a personal computer (PC), a tablet computer, a laptop computer, a netbook, a PDA, a cellular telephone, a smart phone, a mobile device, a wearable device, other smart devices, a web appliance, a network router, a network switch, a network bridge, or any machine capable of executing the instructions 512, sequentially or otherwise, that specify actions to be taken by the machine 502. Further, while only a single machine 502 is illustrated, the term “machine” shall also be taken to include a collection of machines that individually or jointly execute the instructions 512 to perform any one or more of the methodologies of this solution as discussed herein.

The machine 502 may include processors 506, memory 508, and I/O components 504, which may be configured to communicate with each other via a bus 542. In an example of the solution, the processors 506 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an ASIC, a Radio-Frequency Integrated Circuit (RFIC), another Processor, or any suitable combination thereof) may include, for example, a Processor 510 and a Processor 514 that execute the instructions 512. The term “Processor” is intended to include multi-core processors that may comprise two or more independent processors (sometimes referred to as “cores”) that may execute instructions contemporaneously. Although FIG. 5 shows multiple processors 506, the machine 502 may include a single Processor with a single core, a single Processor with multiple cores (e.g., a multi-core Processor), multiple processors with a single core, multiple processors with multiples cores, or any combination thereof.

The memory 508 includes a main memory 516, a static memory 518, and a storage unit 520, both accessible to the processors 506 via the bus 542. The main memory 516, the static memory 518, and storage unit 520 store the instructions 512 embodying any one or more of the methodologies or functions described herein. The instructions 512 may also reside, completely or partially, within the main memory 516, within the static memory 518, within machine-readable medium 522 within the storage unit 520 within at least one of the processors 506 (e.g., within the Processor's cache memory), or any suitable combination thereof, during execution thereof by the machine 502.

The I/O components 504 may include a wide variety of components to receive input, provide output, produce output, transmit information, exchange information, capture measurements, and so on. The specific I/O components 504 that are included in a particular machine will depend on the type of machine. For example, portable machines such as mobile phones may include a touch input device or other such input mechanisms, while a headless server machine will likely not include such a touch input device. It will be appreciated that the I/O components 504 may include many other components that are not shown in FIG. 5. In various example of the solutions, the I/O components 504 may include output components 528 and input components 530. The output components 528 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 530 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example of the solutions, the I/O components 504 may include biometric components 532, motion components 534, environmental components 536, or position components 538, among a wide array of other components. For example, the advanced biometric components 532 of this solution include components to detect expressions related to autism (e.g., hand expressions, facial expressions, vocal expressions, body gestures, or eye-tracking), measure bio-signals indicative of autism (e.g., blood pressure, heart rate, body temperature, perspiration, levels of carbon dioxide/other chemicals in blood work, or brain waves), identify a personal individual characteristics related to autism (e.g., voice identification, retinal identification, facial identification, fingerprint identification, or electroencephalogram-based identification), and the like.

Communication may be implemented using a wide variety of technologies. The I/O components 504 further include communication components 540 operable to couple the machine 502 to a network 524 or devices 526 via respective coupling or connections. For example, the communication components 540 may include a network interface Component or another suitable device to interface with the network 524. In further examples, the communication components 540 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 526 may be another machine or any of a wide variety of peripheral devices (e.g., a peripheral device coupled via a USB).

Moreover, the communication components 540 may detect identifiers or include components operable to detect identifiers. For example, the communication components 540 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 540, such as location via Internet Protocol (IP) geolocation, location via Wi-Fi® signal triangulation, location via detecting an NFC beacon signal that may indicate a particular location, and so forth.

The various memories (e.g., main memory 516, static memory 518, and/or memory of the processors 506) and/or storage unit 520 may store one or more sets of instructions and data structures (e.g., software) embodying or used by any one or more of the methodologies or functions described herein. These instructions (e.g., the instructions 512), when executed by processors 506, cause various operations to implement the disclosed examples of the solutions.

The instructions 512 may be transmitted or received over the network 524, using a transmission medium, via a network interface device (e.g., a network interface component included in the communication position components 538) and using any one of several well-known transfer protocols (e.g., hypertext transfer protocol (HTTP)). Similarly, the instructions 510 may be transmitted or received using a transmission medium via a coupling (e.g., a peer-to-peer coupling) to the devices 526.

As previously mentioned, this solution utilizes machine learning methodology to create a training solution for the data processed by each user and each client processed using this solution. By continuously measuring results of assessment outcomes and comparing it to the scoring algorithm, the present solution improves the predicted Assessment Output 118.

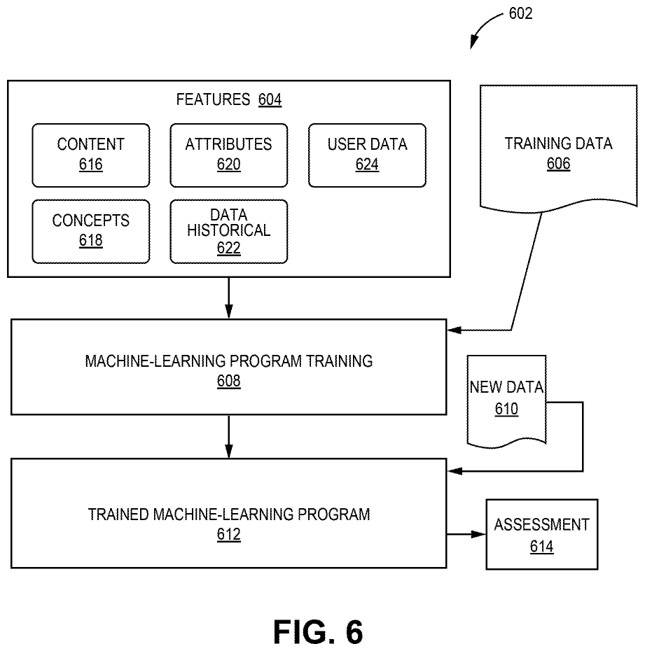

FIG. 6 illustrates training and use of a machine-learning program 602 by the present solution according to some example of the solutions. In some example of the solutions, machine-learning programs (MLPs), also referred to as machine-learning algorithms or tools, are used to perform operations associated with searches, such as job searches.

### Use of Machine Learning Algorithms to Improve Aba Results

By training the solution on a number of collected data sets, the solution improves its recommendations for patients that fall in to defined cohorts. An example of the solution implementing a variety of MLPs is described below. Training of the solution selects combinations of training data comprising the following elements: historical data of ABA treatments, treatment variables, demographics of the patients, including cohort data, demographic of the patients parents, the demographics of the prescribers, the treatments the success of various ABA treatments, the frequency and limitations on which ABA treatments are paid for by insurance, data quality parameters reflecting the confidence and completeness of the data gathered, among other items listed in previous lists. The training of the solution improves the recommendations or inherent knowledge to be presented for the prescriber that are tailored to the patient's particular conditions. In another example of the solution, the MLPs are used to capture and train the solution in real time interaction with the patient, the patient's parents or care takers, and the prescribing clinician and any support staff. Depending on the MLPs incorporated on the mobile devices that may be used for data capture, a parent of the patient may use the functions of the mobile device MLP programs to capture audible, visual or other physical evidence that would be added to the ABA record related to the patient. Suitable privacy and de-identification filters would be applied to any data from any particular client or session to allow ongoing contribution of training data to the training corpus of historical data.

Machine learning is a field of study that gives computers the ability to learn without being explicitly programmed. Machine learning explores the study and construction of algorithms, also referred to herein as tools that may learn from existing data and make predictions about new data. Such machine-learning tools operate by building a model from example training data 606 in order to make data-driven predictions or decisions expressed as outputs or assessments (e.g., assessment 614). Although example of the solutions are presented with respect to a few machine-learning tools, the principles presented herein may be applied to other machine-learning tools.

In some example of the solutions, different machine-learning tools may be used. For example, Logistic Regression (LR), Naive-Bayes, Random Forest (RF), neural networks (NN), matrix factorization, and Support Vector Machines (SVM) tools may be used for classifying or scoring clients and their cohorts.

Two common types of problems in machine learning are classification problems and regression problems. Classification problems, also referred to as categorization problems, aim at classifying items into one of several category values (for example, is this object an apple or an orange?). Regression algorithms aim at quantifying some items (for example, by providing a value that is a real number).

The machine-learning algorithms use features 604 for analyzing the data to generate an assessment 614. Each of the features 604 is an individual measurable property of a phenomenon being observed. The concept of a feature is related to that of an explanatory variable used in statistical techniques such as linear regression. Choosing informative, discriminating, and independent features is important for the effective operation of the MLP in pattern recognition, classification, and regression. Features may be of different types, such as numeric features, strings, and graphs.

In one example of the solution, the features 604 may be of different types and may include one or more of content 616, concepts 618, attributes 620, historical data 622 and/or user data 624, merely for example.

The machine-learning algorithms use the training data 606 to find correlations among the identified features 604 that affect the outcome or assessment 614. In some example of the solutions, the training data 606 includes labeled data, which is known data for one or more identified features 604 and one or more outcomes, such as detecting communication patterns, detecting the meaning of the message, generating a summary of a message, detecting action items in messages detecting urgency in the message, detecting a relationship of the user to the sender, calculating score attributes, calculating message scores, etc.

With the training data 606 and the identified features 604, the machine-learning tool is trained at machine-learning program training 608. The machine-learning tool appraises the value of the features 604 as they correlate to the training data 606. The result of the training is the trained machine-learning program 612.

When the trained machine-learning program 612 is used to perform an assessment, new training new data 610 is provided as an input to the trained machine-learning program 612, and the trained machine-learning program 612 generates the assessment 614 as output.

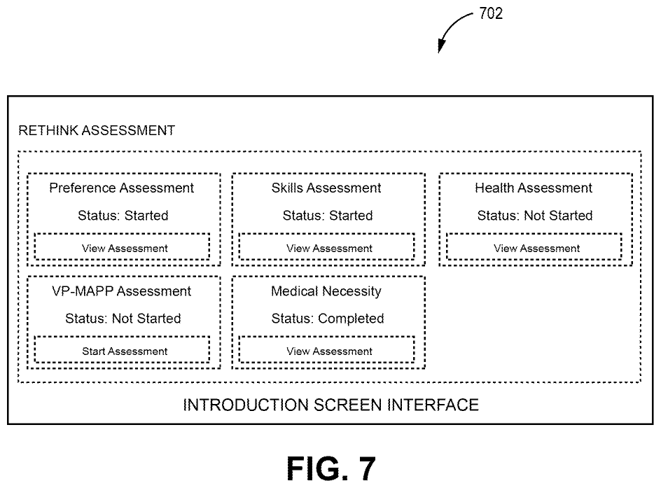

The Introduction Screen User Interface 702 is presented in FIG. 7 showing basic choices as presented in the Assessment Flow 100 FIG. 1. This allows the user to select the type of assessment to be performed.

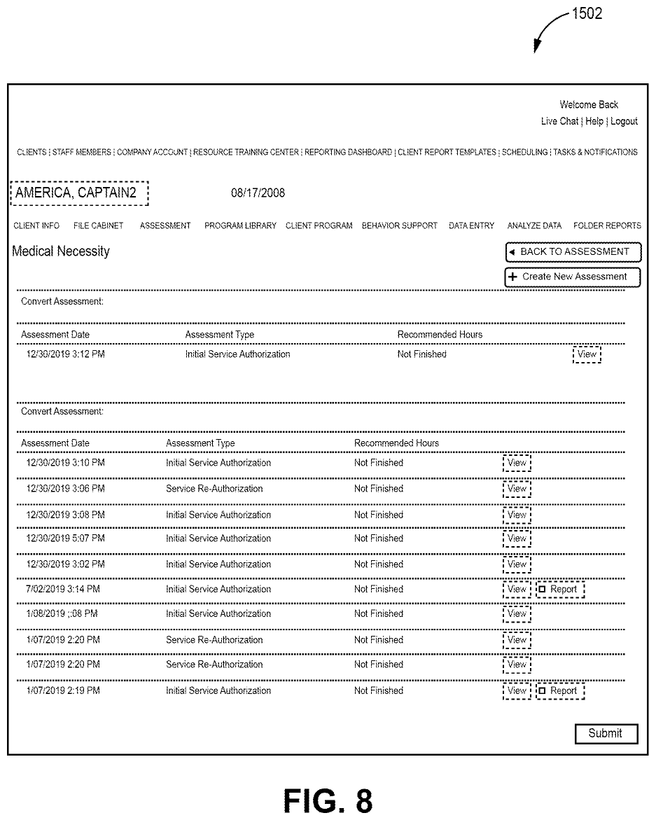

Open Assessment User Interface 802 as shown in FIG. 8 tracks all of the open assessments being made by the solution user. This interface example allows the user to quickly browse as to which assessment has a work requirement.

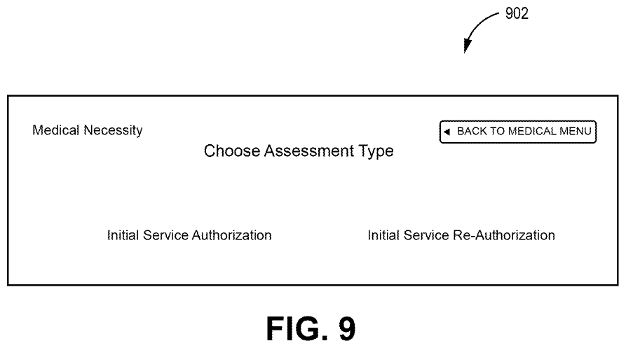

Different assessment choices are available in Assessment Choice User Interface 902 shown in FIG. 9. Assessment authorization varies dependent upon a service being initialized or a service being reauthorized.

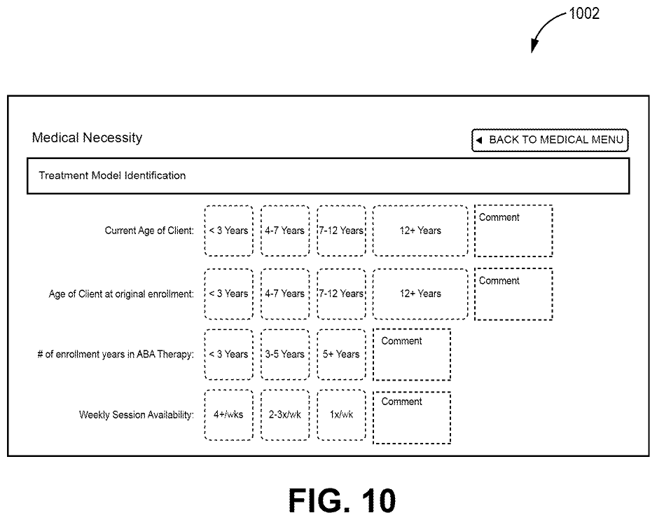

FIG. 10 identifies the variable models in Treatment Model Input User Interface 1002 showing dimensions of Age, length of treatment, Age of Client at enrollment, and the periodicity of treatments available.

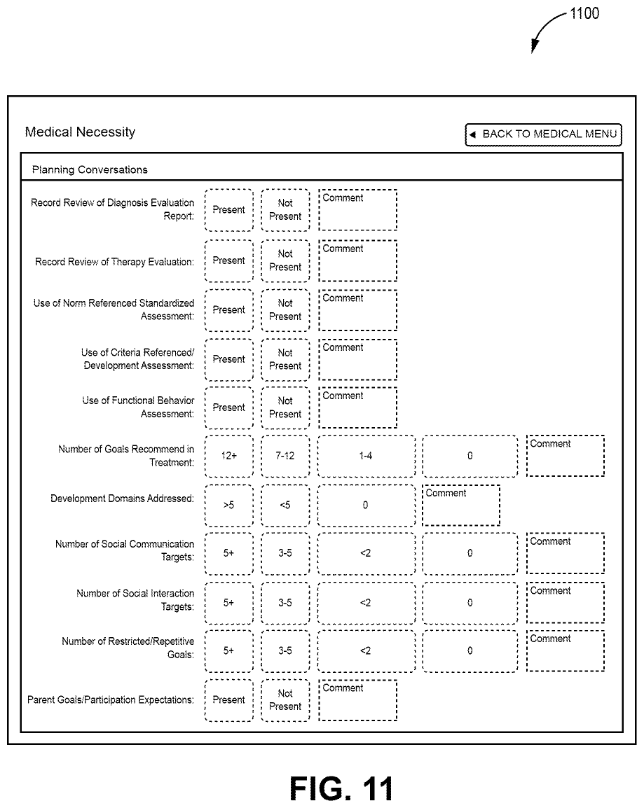

FIG. 11 shows many of the planning considerations inherent in an ABA model by Planning Data User Interface 1100. This interface exemplifies the complexity of managing and recording ABA treatments.

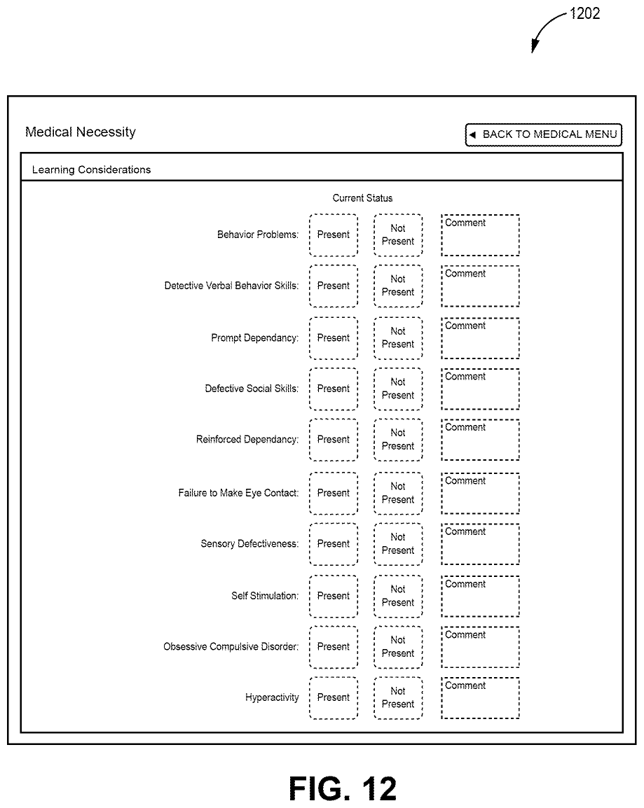

FIG. 12 demonstrates the multifactor learning considerations in executing the Current Status User Interface 1202. This interface also captures the various biometric components 532 that may be used to augment a professional psychologist evaluation for an ABA regimen.

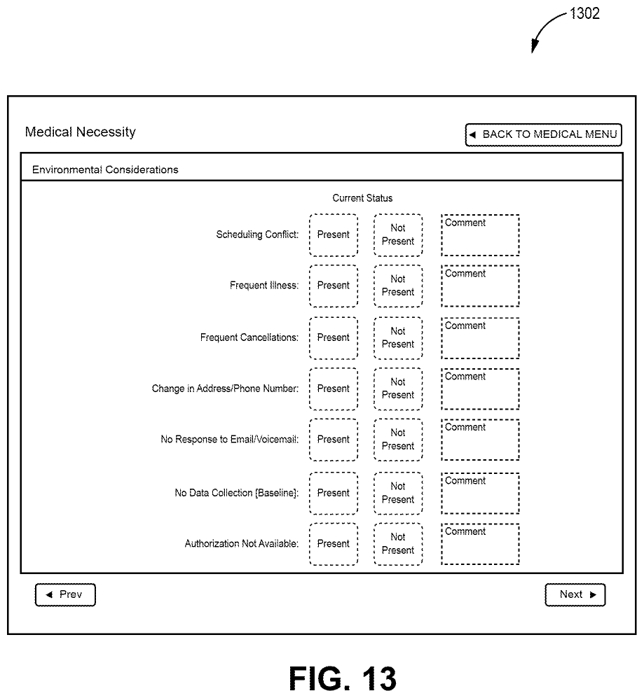

FIG. 13 shows the Environmental Considerations User Interface 1302 captures the current environmental conditions for the subject considered for the ABA model to be engaged on the subject.

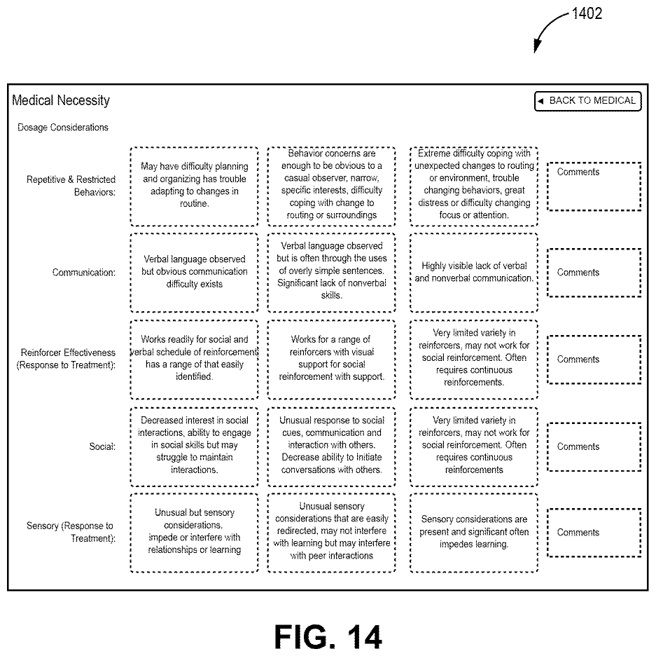

Clinical Data User Interface 1402 establishes the dosage baselines and modifications to the baseline in FIG. 14.

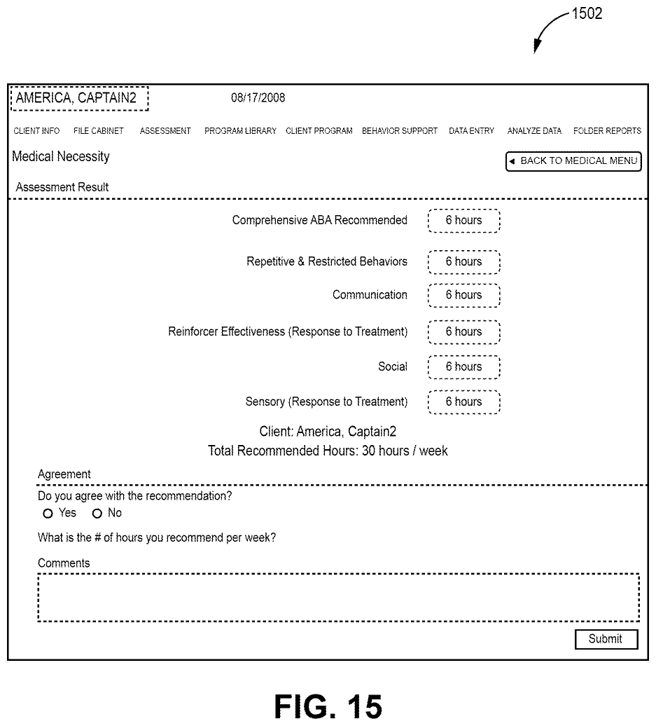

FIG. 15 is a Result and Recommendation User Interface 1502 example of the solution.

This a view of a Comprehensive ABA recommendation prescribing treatment in hours across five categories. This interface also captures agreement of the clinician with the recommendation. This is the first step in the feedback of improving the recommendation result.

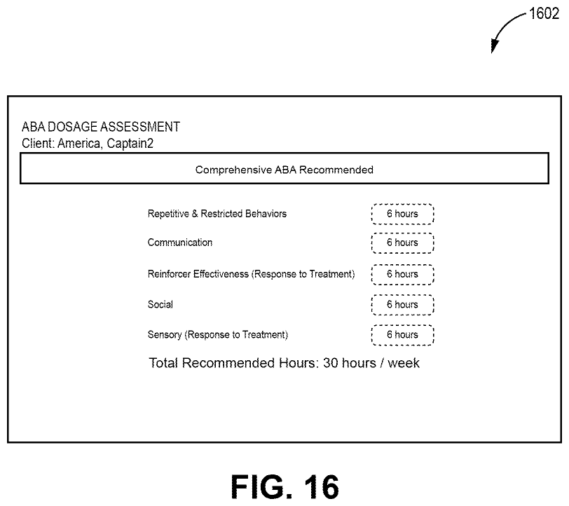

FIG. 16 shows Results Summary User Interface 1600 shows an example of the solution showing a separate example of a result summary that would be shown to a payer or guardian of the patient in a Comprehensive ABA recommendation.

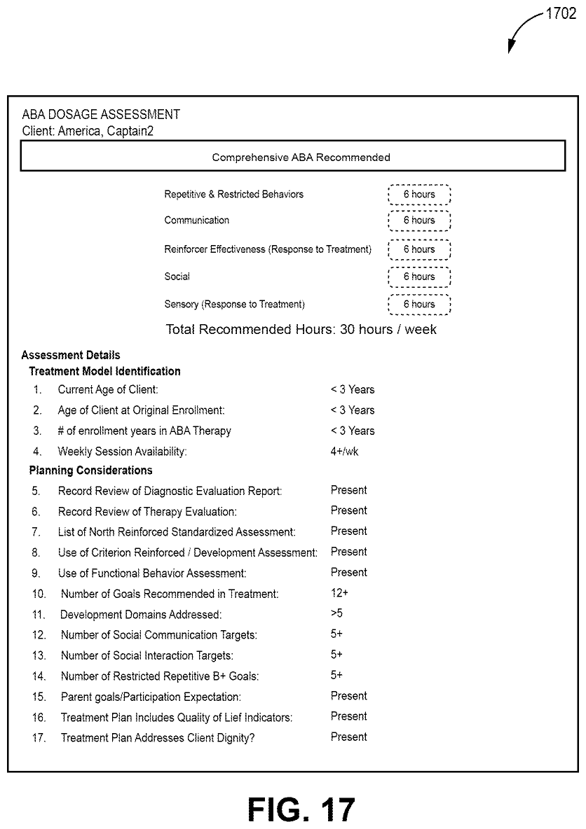

Comprehensive Report User Interface 1702 example is partially shown in FIG. 17.

This interface summarizes all of the unique considerations captured by the Current Status User Interface 1200 solution for the individual in concordance with the views of the practitioner in a manner most appropriate for the Payer.

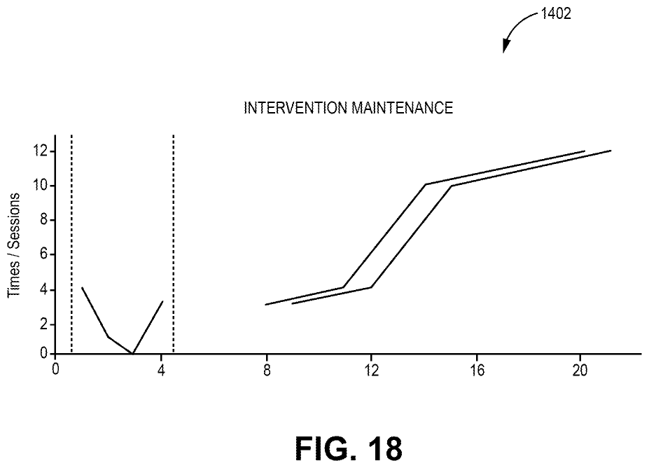

A charting interface example 1802 is shown in FIG. 18 showing the progression of intervention and the charting of ABA therapy targets and the execution of the actual therapy.

This interface view can be used to represent various expected ABA therapy goals and inputs/transformations/outputs.

The evidence supporting the use of ABA in children with autism up to the age of twelve is compelling. While limitations in research are present regarding the effects of ABA as it pertains to population health management, the overwhelming support for covering these services remains. Additionally, in the recent years there has been a strong effort to remove funding-based treatment decisions done on behalf of managed care organizations and increase access to proper dosage recommendation by the clinician influenced best practice standards.

The challenge in utilization review may simply be a complex language barrier between the treatment providers and managed care organizations For example, the behavior analyst has a priority to their individual clients whereas the managed health care organizations have a priority to whole patient populations. A collaborative effort to understand one another's fiscal, clinical and patient outcome needs will continue to be of great importance. This also presents us with a unique opportunity to create clinical job aids pertaining to treatment intensity recommendations that may also serve to promote standards of care amongst treating providers nationwide and drive the continued research on the individual features of ABA treatment for individuals with autism of all ages.

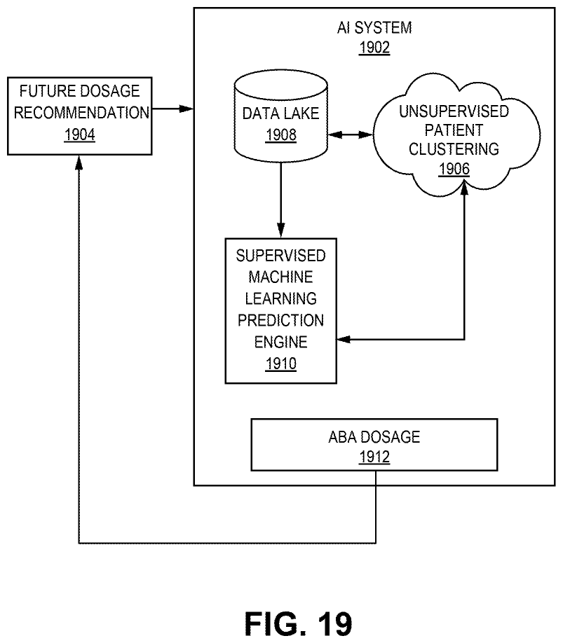

FIG. 19 illustrates an example routine for an AI assisted Future Dosage 1904 from an AI System 1902. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method in describes a variant of the solution for calculation of an ABA Future Dosage 1904. The system comprises an AI System 1902, an Unsupervised Patient Clustering 1906, a Data Lake 1908, a Supervised Machine Learning Prediction Engine 1910, and an ABA Dosage 1912 The Future Dosage 1904 is designed to improve the accuracy and efficiency of medical dosing by utilizing artificial intelligence and machine learning technologies. This dosing AI machine integrates multiple components to aim for a reduction in errors and improved patient outcomes.

The AI System 1902 collects and analyzes real-time data. It integrates with electronic health records and other machine or human data sources, continuously updating patient information. The AI algorithms perform calculations to determine optimal dosages for a range of behavioral health issues. These calculations take into account multiple variables such as patient age, weight, medical history, mental health history, treatment regimes, and current health conditions.

The Unsupervised Patient Clustering 1906 groups patients into categories based on common characteristics or treatment profiles. This is done without human intervention. By clustering similar patients together, the system aims to better predict the effectiveness of different treatment plans. Generally these clusters can be called cohorts. In the context of patient clustering, a cohort refers to a group of patients who share specific characteristics, medical histories, or treatment responses. The purpose of creating such cohorts is often to analyze and understand patterns that might not be apparent when examining individual cases.

By clustering patients into cohorts, healthcare providers and researchers can more effectively deliver individually tailored, real-time adaptive interventions that maximize patient outcomes.

Cohort clustering can be based on various criteria, including but not limited to:
Age
Gender
Diagnosis
Medical history
Genetic markers
Social determinants of health
Behavioral patterns
Response to treatment

This could allow healthcare providers to tailor medication plans to individual patient needs, potentially reducing side effects and increasing the effectiveness of treatments. The Data Lake 1908 is a database that stores all the data collected and analyzed by the system. It includes everything from patient records to dosage recommendations. The Data Lake is built for high-speed data retrieval and is meant to provide healthcare providers with up-to-date information for clinical decision-making.

The AI System 1902 includes a Supervised Machine Learning Prediction Engine 1910, which uses labeled data to train itself to make dosage recommendations. The engine learns from both successful and unsuccessful treatments to adjust its algorithms over time. The objective is to improve the accuracy and reliability of the dosage recommendations continually. The ABA Dosage 1912 module applies principles from Applied Behavior Analysis (ABA) to medication and other types of treatment dosing. This approach aims to evaluate the interaction between behavior and the environment. It's hypothesized that considering behavioral factors could lead to more personalized treatment plans and possibly better patient outcomes.

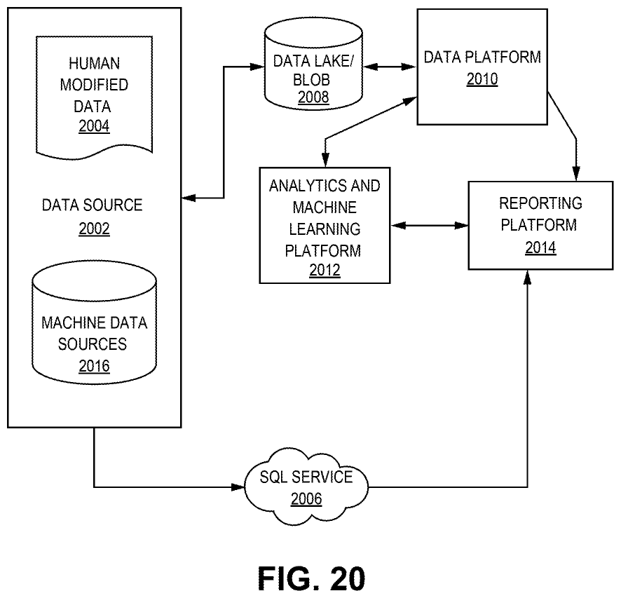

FIG. 20 is an example of data sources of an ABA platform variant of the present solutions The system comprises a Data Source 2002, a Human Modified Data 2004, a SQL Service 2006, a Data Lake/Blob 2008, a Data Platform 2010, an Analytics and Machine Learning platform 2012, a Reporting Platform 2014, and a Machine Data Sources 2016. And Data Source 2002: The primary origin of raw data, the Data Source 2002 can be any entity that generates or collects information. This could range from web applications, sensors, user interactions, to traditional databases. This raw data, in its unprocessed form, is the foundational building block for all subsequent operations and interactions.

Human Modified Data 2004: Once data is collected from the source, there might be instances where human intervention is needed for rectifications, additions, or modifications. The Human Modified Data 2004 represents this manually altered data. It's crucial that this component interacts flawlessly with the primary data source to ensure that changes made reflect accurately and maintain data integrity.

SQL Service 2006: A pivotal player in the data ecosystem, the SQL Service 2006 offers structured querying capabilities to retrieve, manipulate, and manage data. Whether it's fetching data from the original source or accessing human-modified data, the SQL service ensures that data can be accessed in a structured, efficient, and reliable manner. Its interaction with the Human Modified Data 2004 also guarantees that any manual modifications are queryable and integrated into the overall data flow.

Data Lake/Blob 2008: With the explosion of data in both volume and variety, there arose a need for flexible, scalable, and diverse storage solutions. The Data Lake/Blob 2008 is that solution, providing a repository for storing vast amounts of raw data in its native format, be it structured, semi-structured, or unstructured. By directly interacting with the SQL Service 2006, it ensures that data, irrespective of its source, can be stored and accessed without constraints.

Data Platform 2010 The Data Platform 2010 acts as the central hub that orchestrates the movement, transformation, and storage of data. It interacts with the Data Lake/Blob 2008 to fetch data, utilizes the SQL Service 2006 to query and transform the data, and ensures that the Human Modified Data 2004 is seamlessly integrated. The platform embodies the infrastructure and tools required to handle, process, and route data to various other components. An example: Azure Synapse Analytics, formerly known as Azure SQL Data Warehouse, is an integrated analytics service provided by Microsoft Azure.

Analytics and Machine Learning platform 2012: With data at its fingertips, this platform is where advanced computations and predictive analytics happen. Extracting data from the Data Platform 2010, the Analytics and Machine Learning platform 2012 applies algorithms, statistical models, and machine learning techniques to draw insights, make predictions, or even automate decision-making processes. Its interaction with Machine Data Sources 2016 ensures that machine-generated data can also be used for analytical purposes, enriching the overall analysis. An example of this platform would be Databricks is a cloud-based platform for big data analytics and machine learning.

Reporting Platform 2014: After analyzing data, the insights drawn need to be presented in a comprehensible manner for stakeholders. The Reporting Platform 2014 does just that. By sourcing data from the Analytics and Machine Learning platform 2012, it generates visualizations, dashboards, and reports that condense vast amounts of information into digestible formats. Its tight integration ensures that insights are timely, accurate, and actionable.

Machine Data Sources 2016: In today's era of IoT and automation, machines generate a staggering amount of data. The Machine Data Sources 2016 represent this data, encompassing logs, sensor readings, and automation outputs. This data, when funneled into the Analytics and Machine Learning platform 2012, offers unparalleled insights into machine operations, efficiency, and predictive maintenance.

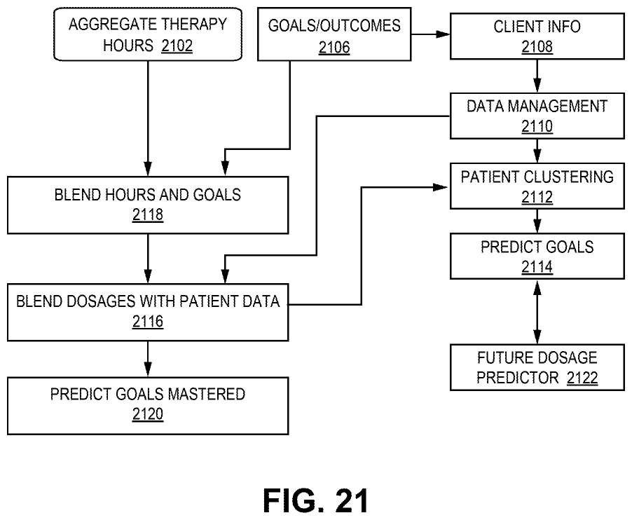

FIG. 21 illustrates an example routine for the AI System 1902. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method includes at Goals/Outcomes 2106 where the goals or outcomes for a class of patient or a cluster of patients are defined and stored.

According to some examples, the method includes at Client Info 2108 where various patient data, and other relationships of individual patients with various providers are defined and stored.

According to some examples, the method includes at Data Management 2110 is a preclustering data management step to allow client data to preset for a Patient Clustering 2112 step.

According to some examples, the method includes at Patient Clustering 2112. This step clusters Client Info 2108 into unique patient clusters to allow the Analytics and Machine Learning platform 2012 to manage those clusters in light of treatment options.

According to some examples, the method includes at Predict Goals 2114 where current goals and progress of the patient are correlated via, processor to recommend changes to goals if the Patient is performing in a manner different than previously projected.

According to some examples, the method includes at Blend Dosages with Patient Data 2116. This step merges dosage levels with various Patient data for further analytics.

According to some examples, the method includes at Blend Hours and Goals 2118. Similarly, a step to blend dosage hours and realized Patient goals is performed here to allow for further analytics.

According to some examples, the method includes at Predict Goals Mastered 2120. This uses machine learning processes to digest aggregated data on patients and make machine generated predictions of the efficacy of a certain prescribed therapy, with a certain type of patient and a machine predicted outcome of that therapy for that type of patient. The system also comprises an Aggregate Therapy Hours 2102 step where the patient treatment hours is aggregated and a Future Dosage Predictor 2122 step where a future treatment is predicted given a patient's current state and their current progress with the present therapy.

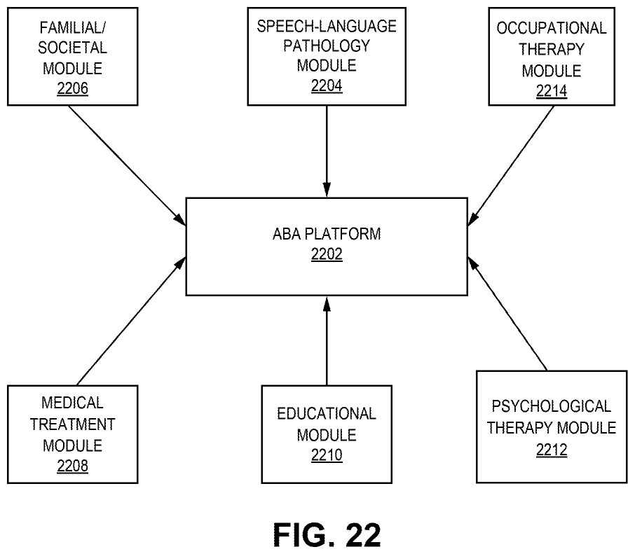

FIG. 22 describes the various segmented components of the system that comprises an ABA Platform 2202, a Speech-Language Pathology Module 2204, a Familial/Societal Module 2206, a Medical Treatment Module 2208, an Educational Module 2210, a Psychological Therapy Module 2212, and an Occupational Therapy Module 2214.

ABA Platform 2202: At its core, the ABA Platform 2202 is designed to understand and modify socially significant behaviors of the patient . . . Through a series of structured interventions and data recording, the platform ensures that the patient is treated for desired behaviors while reducing undesired behaviors. Caregivers can track their patient's progress, modify interventions, and interact with other healthcare processes in an integrated manner.

Speech-Language Pathology Module 2204: Interacting with ABA Platform 2202, the Speech-Language Pathology Module 2204 process focuses on diagnosing, treating, and preventing speech, language, voice, and fluency disorders. A patient's ABA goals may overlap with their speech-language goals. For instance, a child might be working on both verbal communication skills in ABA therapy and articulation in speech therapy. The integrated platform ensures that caregivers and therapists can collaborate, ensuring the strategies align and support each other.

Familial/Societal 2206 Process: Understanding the familial and societal contexts is crucial in implementing effective ABA interventions. The Familial/Societal Module 2206 process assesses the patient's family dynamics, cultural nuances, and societal roles either by a series of interviews or statistical predictors. By integrating with the ABA Platform 2202, it ensures that the interventions are contextual, culturally sensitive, and can be effectively implemented in the patient's natural environment.

Medical Treatment Module 2208: Certain patients undergoing ABA therapy might also have concurrent medical treatments. The Medical Treatment Module 2208 process outlines the patient's medical regime, including medication, physical check-ups, and other medical procedures. By interacting with the ABA Therapy Platform 2202, it ensures that the ABA interventions do not interfere with the patient's medical regimen and vice versa. Caregivers are provided with a comprehensive understanding of the patient's health and behavioral needs.

Patient and Caregiver Educational Module 2210: The Educational Module 2210 forms a critical component of the therapy journey. The Patient and Caregiver Educational Module 2210 process is designed to provide both the patient and their caregivers with the knowledge, skills, and resources necessary for effective therapy. This can range from understanding the principles of ABA to learning specific intervention techniques. Integrated with the ABA platform, it ensures that the educational material is tailored to the patient's specific ABA goals and progress.

Psychological Therapy Module 2212: Some patients might require concurrent psychological intervention the process used by the Psychological Therapy Module 2212 caters to the patient's mental health needs, from cognitive-behavioral therapy to counseling sessions. Interacting with the ABA Therapy Platform 2202, this process ensures that the patient's ABA goals and psychological treatment strategies complement and do not counteract each other. Caregivers can have a holistic view of the patient's mental and behavioral health journey.

Occupational Therapy Module 2214: Occupational therapy aims at equipping patients with the skills necessary for daily living and independence. The Occupational Therapy Module 2214 process, when integrated with the ABA platform, focuses on areas of overlap between the two. For instance, a patient working on fine motor skills in occupational therapy might also have related behavioral goals in ABA. Caregivers and therapists from both disciplines can collaborate, ensuring that the strategies align and the patient receives a well-rounded intervention.

The evidence supporting the use of behavior analysis in children with autism up to the age of twelve is compelling. While limitations in research are present regarding the effects of behavior analysis as it pertains to population health management, the overwhelming support for covering these services remains. Additionally, in recent years, there has been a strong effort to remove funding-based treatment decisions done on behalf of managed care organizations and increase access to proper dosage recommendation by the clinician influenced best practice standards.

The challenge in utilization review may simply be a complex language barrier between the treatment providers and managed care organizations For example, the behavior analyst has a priority to their individual clients whereas the managed health care organizations have a priority to whole patient populations. A collaborative effort to understand one another's fiscal, clinical and patient outcome needs will continue to be of great importance. This also presents the mental and behavioral health community with a unique opportunity to create clinical job aids pertaining to treatment intensity recommendations that may also serve to promote standards of care amongst treating providers nationwide and drive the continued research on the individual features of ABA treatment for individuals with autism of all ages.

The above description includes references to the accompanying drawings, which form a part of the detailed description. The drawings show, by way of illustration, specific examples of the solutions in which the invention can be practiced. These variants of the solutions are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise.

Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a Computer-Readable Medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other variants of the solutions can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed variant of the solution. Thus, the following claims are hereby incorporated into the Detailed Description as examples or variants of the solutions, with each claim standing on its own as a separate variant of the solution, and it is contemplated that such variant of the solutions can be combined with each other in various combinations or permutations. The scope of the invention should be determined with reference to the appended claims, along with the full scope of equivalents to which such claims are entitled.

What is claimed is:

1. A computer-implemented automated monitoring and behavioral health treatment recommendation method comprising:
receiving a set of data from a patient sensor and a set of data from a caregiver for a patient;
computing, via a processor, a behavioral health dosage for the patient;
correlating, via a processor, the behavioral health dosage to a corpus of behavioral health dosage treatment data;
and calibrating the behavioral health dosage, via processor; in light of a cohort of similar patient behavioral health dosages and;
measuring a progress metric towards a set treatment goal of the patient, via the processor.

2. The computer-implemented automated monitoring and behavioral health treatment recommendation method of claim 1 further comprising receiving additional patient data from a behavioral health treatment ecosystem using a set of data from at least one therapy module.

3. The computer-implemented automated monitoring and behavioral health treatment recommendation method of claim 2 wherein the behavioral health treatment ecosystem extends to a Virtual Medical Home instance.

4. The computer-implemented automated monitoring and behavioral health treatment recommendation method of claim 3 wherein the Virtual Medical Home instance further comprises a set of sensors that measure a patient's biometrics in real-time.

5. The computer-implemented automated monitoring and behavioral health treatment recommendation method of claim 4 wherein the patient's biometrics are correlated to the cohort of similar patient behavioral health dosages.

6. The computer-implemented automated monitoring and behavioral health treatment recommendation method of claim 5 wherein the patient's biometrics are captured by a wearable sensor.

7. The computer-implemented automated monitoring and behavioral health treatment recommendation method of claim 6 further comprising that based on the patient's real-time biometric measurements, a change in the patient's behavioral health dosages is recommended to a caregiver of the patient.

8. The computer-implemented behavioral health assessment for behavioral health therapy method of claim 1 further comprising receiving data from the behavioral health treatment ecosystem using a set of data from at least one therapy module and at least one familial/societal module

9. The computer-implemented behavioral health assessment for behavioral health therapy method of claim 8 wherein the at least one therapy module is selected from a group of (a medical therapy module, an occupational therapy module, a speech-language-pathology module, or a psychological therapy module)

10. The computer-implemented behavioral health assessment for behavioral health therapy method of claim 1 further comprising a patient cluster data set that is correlated, via a processor, to an AI predicted outcome related to a recommended behavioral health treatment.

11. The computer-implemented behavioral health assessment for behavioral health therapy method of claim 10 wherein, the predicted outcome is correlated across a cluster of patent treatment data.

12. The computer-implemented behavioral health assessment for behavioral health therapy method of claim 11 wherein the predicted outcome is used to adjust a patient's dosage in real-time.

13. One or more non-transitory computer readable media comprising instructions that, when executed with a computer system configured to execute instructions with binary data, cause the computer system to at least:
receive, by a computer system, a set of binary data correlated to an individual behavioral condition captured via a sensor connected to the individual;
correlate, by the computer system, that the set of binary data representative of an behavioral health dosage for the patient;
execute, by the computer system, suggestion of the behavioral health dosage for the patient further comprising a set of treatment goals;
recalibration, by the computer system, a progress correlation between the set of binary data correlated to an individual behavioral conditions and the set of treatment goals; and
recommend, by the computer system, a recommendation a change in the behavioral health dosage for the patient to enhance the progress correlation.

14. The non-transitory computer readable media of claim 13 further comprising the transmission of additional patient data from a behavioral health treatment ecosystem.

15. The non-transitory computer readable media of claim 14 wherein the behavioral health treatment ecosystem further comprises a group of modules selected from a group of (a medical therapy module, an occupational therapy module, a speech-language-pathology module, or a psychological therapy module).

16. The non-transitory computer readable media of claim 15 wherein the behavioral health treatment ecosystem extends a Virtual Medical Home into the patient's place of residence.

17. The non-transitory computer readable media of claim 16 further comprising the use of a wearable sensor on the patient to capture a set of the patient biometric data.

18. The non-transitory computer readable media of claim 17 further comprising a measurement of a patient's eye-track as part of the set of biometric data
